(19) United States
(12) Patent Application Publication
(10) Pub. No.: US 2015/0112383 A1
(43) Pub. Date: Apr. 23, 2015

(54) CLOSURE DEVICE

(71) Applicant: Cook Medical Technologies LLC, Bloomington, IN (US)

(72) Inventors: Sara Marie Sherman, Lafayette, IN (US); Steven J. Charlebois, Lafayette, IN (US)

(73) Assignee: Cook Medical Technologies LLC, Bloomington, IN (US)

(21) Appl. No.: 14/058,897

(22) Filed: Oct. 21, 2013

(51) Int. Cl. A61B 17/00

(52) U.S. Cl. CPC A61B 17/0057
USPC 606/213

(57) ABSTRACT

A closure device for a body passageway of a patient includes a plug member formed from an extracellular matrix material. The plug has a compressed condition, and is modifiable therefrom to an expanded condition for occluding the body passageway. A plurality of first frame members is disposed along a first side of the plug. The first frame members are aligned in a first position relative to the plug when the plug is in the compressed condition, and movable therefrom to a second position for occlusion of the passageway. A plurality of second frame members is disposed generally along a second side of the plug. The second frame members are aligned in a first position relative to the plug when the plug is in the compressed condition, and movable therefrom to a second position for occlusion of the passageway.

### BACKGROUND OF THE INVENTION

1. Technical Field

The present invention relates to a device and method for closure of a body passageway in a patient. More particularly, the invention relates to a percutaneously-introduced device and method for closure of a patent foramen ovale (PFO).

2. Background Information

In the fetal heart, there is a small opening, referred to as the foramen ovale, in the septum between the right and left atria. In the unborn fetus, this opening allows blood to bypass the lungs, as fetal blood is oxygenated by the lungs of the mother. This opening normally closes within the first year after birth, and oxygenation is carried out through the baby's own lungs.

In some cases, however, this opening (the foramen ovale) remains patent. In this instance, the baby's oxygenated blood is diluted by unoxygenated venous blood that passes through the patent foramen ovale. Babies with this condition often have very little energy, are cyanotic (blue coloration), and do not progress well after birth. In order to repair this defect, the opening can be closed by surgical methods, or by newer percutaneous methods.

In recent years, physicians have also discovered that in a large percentage of adults, estimated at about 25% of the adult population, the foramen ovale has not completely sealed, and remains as a small patent foramen ovale. In these adults, there is still some leakage across the septum through the remnant foramen ovale. Although such leakage is not always problematic, the leakage can be aggravated upon certain types of strain or stress on the body. Intermittent leakage of blood through the PFO has been linked to migraine headaches and other maladies. In addition, a PFO is suspected as being a passageway for blood clots. Passage of clots through the opening can lead to a stroke or a transient ischemic attack (TIA).

The leaking, or patent, foramen ovale does not result from the same physiological structure as an atrial septal defect (ASD). An ASD is normally a definable hole that extends through the septum. Such holes can be occluded by passing known occluder devices through the hole, such that the devices anchor on each side of the septum to form a seal. Unlike the definable hole that forms an ASD, the foramen ovale is a small channel or slot-type structure that is defined by the septum primum and the septum secundum. With a PFO, the septum primum and septum secundum normally overlap to a certain degree, and are not fused together as in the normal case. As a result, small amounts of blood may leak through the passageway.

Currently available ASD repair devices are not well suited for repair of a PFO. As stated, ASD repair devices normally comprise an occluder-type structure that is extended through the septum hole that comprises the defect to seal the opening. However, with a PFO, the openings on each side of the septum are offset, and not in line with each other (i.e., not directly across from each other). The leakage path is under a flap, and through a narrow passageway, rather than a defined hole. Thus, it is not generally sufficient to merely provide a plug for a hole, as in conventional ASD repair.

Open heart surgical methods have been used for PFO repair. Such methods normally entail breaking open the chest cavity, and cutting into the heart muscle. The septum secundum is then sutured or otherwise attached to the septum primum, in a manner such that the passageway is closed. Although generally effective for closing the PFO, such methods are intrusive, costly, and require an extended recovery period for the patient.

Recently percutaneous methods have been developed for repair of a PFO. These methods utilize conventional percutaneous entry techniques, such as the Seldinger technique, for passing a catheter through a vessel into the right atrium of the heart. Numerous mechanical closure devices have been utilized for percutaneous repair of atrial septal defects. These devices potentially allow patients to avoid the side effects often associated with anticoagulation therapies and the risks of invasive surgery.

However, devices for treating heart defects, such as PFO and other atrial and ventricular septal heart defects have their share of drawbacks. The complex anatomical features of PFOs present a challenge to a one size fits all approach. The septum secundum is often thicker than the septum primum and exhibits limited mobility and compliance. Failure of these two structures to fuse creates the tunnel-like PFO. The distance of the nonfusion between the two septa determines the particular size of the PFO, which must be considered in the design of a device targeting PFOs.

Conventional nonsurgical closure devices are often technically complex, bulky, have a high septal profile, low radiopacity, and an inability to provide immediate closure. Additionally, many of the devices have a geometry which tends to prevent the device from remaining flat against, or within, the defect once deployed. The varying passageway geometries often require multiple sized devices. Additionally, conventional closure devices are often difficult to deploy or reposition, often require replacement or repositioning, and may require relatively large delivery catheters. Further disadvantages of such nonsurgical closure devices include possible complications resulting from fractures of the components, conduction system disturbances, perforations of heart tissue, and residual leaks.

Accordingly, there is a need for an improved closure device capable of rapidly and securely closing body passageways, such as a patent foramen ovale. The device should be of minimal mechanical and operational complexity, and be capable of closing the body passageway with minimal trauma to the patient.

### SUMMARY

The shortcomings of the prior art are addressed by the closure device described herein. In one embodiment, a device for closure of a body passageway of a patient includes a plug member comprising an extracellular material, and having a first side and a second side. The plug member has a compressed condition, and is modifiable therefrom to an expanded condition for occluding the body passageway. A plurality of first frame members is disposed generally along the first plug member side. The first frame members are aligned in a first position relative to the plug member when the plug member is in the compressed condition, and movable therefrom to a second position. A plurality of second frame members is disposed generally along the second plug member side. The second frame members are aligned in a first position relative to the plug member when the plug member is in the compressed condition, and movable therefrom to a second position.

In another embodiment, a system for closure of a patent foramen ovale of a patient includes a plug member, and a plurality of first and second frame members engaged with the plug member and extendable therefrom. The plug member comprises an extracellular matrix material having a compressed condition for deployment along the patent foramen ovale and modifiable therefrom to an expanded condition. The first frame members are disposed generally along a first side of the plug member and aligned in a first position relative to the plug member in the compressed condition. The first frame members are movable therefrom into a second position relative to the plug member in the expanded condition. The second frame members are disposed generally along a second side of the plug member and aligned in a first position relative to the plug member in the compressed condition. The second frame members are movable therefrom into a second position relative to the plug member in the expanded condition. A delivery device has a lumen for receiving the closure device when the plug member is in the compressed condition, and the first and second frame members are in the first position. The closure device is deployable from the delivery device for expansion of the plug member to the expanded condition, and for movement of the plurality of first and second frame members to the second position, such that the expanded plug member is positionable along the passageway. The first frame members are positionable in the second position along a first side of the septum primum and the septum secundum of the patient, and the second frame members are positionable in the second position along a second side of the septum primum and the septum secundum.

In yet another embodiment, a method for closure of a patent foramen ovale of a patient is provided. The foramen ovale is defined by a passageway in the heart of the patient between the septum primum and septum secundum. A closure device is positioned in the lumen of a delivery device. The closure device comprises a plug member, and a plurality of first and second frame members engaged with the plug member and extendable therefrom. The plug member comprises an extracellular matrix material having a compressed condition when positioned in the delivery device lumen. The first frame members are disposed generally along a first side of the plug member and aligned in a first position relative to the plug member in the delivery device lumen. The second frame members are disposed generally along a second side of the plug member and aligned in a first position relative to the plug member in the delivery device lumen. The delivery device having the closure device positioned therein is introduced into the right atrium of the heart of a patient. The delivery device and closure device are advanced through the patent foramen ovale such that a distal tip of the delivery device is positioned in the left atrium of the patient. Deployment of the closure device from a distal end of the delivery device is initiated such that the first frame members are deployed in the left atrium in the first position, and self-movable therefrom to a second position. The first frame members are aligned in the second position such that a first set of the first frame members abuts the septum primum and a second set of the first frame members abuts the septum secundum in the left atrium. The delivery device is withdrawn relative to the closure device such that the plug member is deployed intermediate the septum primum and the septum secundum, whereupon the plug member is movable from the compressed condition to an expanded condition in the passageway. The delivery device is further withdrawn relative to the closure device such that the second fame members are deployed from the delivery sheath in the right atrium in the first position, and self-movable therefrom to a second position. The second frame members are aligned in the second position such that a first set of the second frame members abuts the septum primum and a second set of said first frame members abuts the septum secundum in the right atrium.

### BRIEF DESCRIPTION OF THE DRAWINGS

FIG. 1 is an illustration of the internal portions of the heart;

FIG. 1A is an enlarged view of a portion of FIG. 1, illustrating the location of a patent foramen ovale;

FIG. 2 is a perspective view of a closure device for a body passageway according to one embodiment of the present invention;

FIG. 3 is a side view of the closure device of FIG. 2 prior to loading into a delivery catheter;

FIG. 3A is a side view of another example of a closure device

FIG. 4 is a view showing the device of FIG. 3 compressed and loaded into the delivery catheter;

FIGS. 5A-5F are views illustrating the introduction and placement of a closure device in a patent foramen ovale of a patient;

FIG. 6 is a side view of a template that may be utilized in forming the closure device; and

FIG. 7 is a side view of the template of FIG. 6 illustrating the formation of frame members for the closure device.

### DESCRIPTION OF PREFERRED EMBODIMENTS

For purposes of promoting an understanding of the present invention, reference will now be made to the embodiments illustrated in the drawings, and specific language will be used to describe the same. It is nevertheless to be understood that no limitation of the scope of the invention is thereby intended. The figures are not all drawn to the same scale to avoid obscuring the details of the finer structures. The following detailed description of the preferred embodiments will make clear the preferred arrangement, size relationships and manner of using the components shown herein.

The present invention relates to a device and method for closure of a body passageway, or opening, such as a patent foramen ovale (PFO). As used herein, the terms “passageway”, “body passageway”, “opening”, and “body opening”, are interchangeably used to refer to a body passageway, opening, aperture, canal, channel, conduit, duct, and the like, including but not limited to a PFO, body valve opening, blood vessel, vessel puncture, bile duct, and the like.

In the following discussion, the terms “proximal” and “distal” will be used to describe the opposing axial ends of the closure device, as well as opposing axial ends of various component features of the device. The term “proximal” is used in its conventional sense to refer to the end of the device (or component) that is closest to the operator during use or insertion of the device. The term “distal” is used in its conventional sense to refer to the end of the device (or component) that is initially inserted into the patient, or that is closest to the patient during use or insertion.

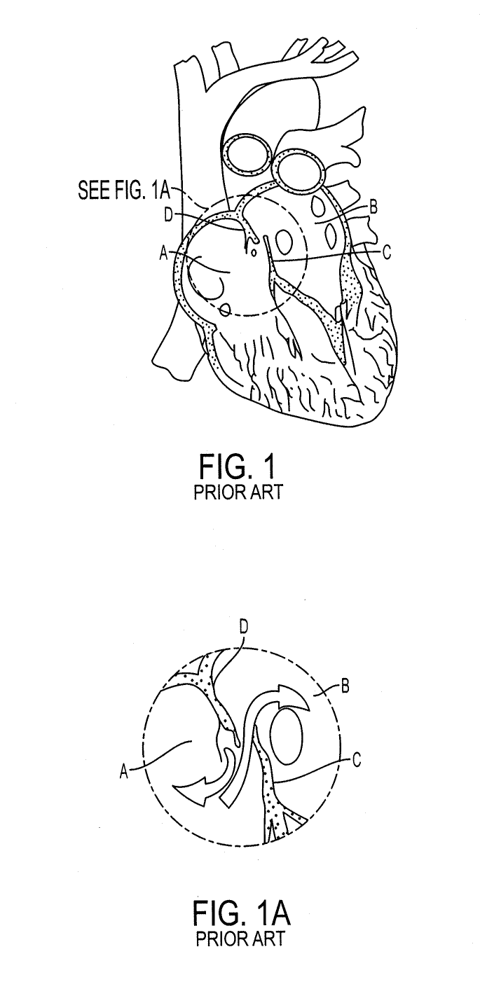

FIGS. 1 and 1A illustrate the internal portions of the heart, including the right atrium A and left atrium B. The septum that separates the right atrium and the left atrium comprises a septum primum C and a septum secundum D. Septum primum C and septum secundum D define a channel (shown by the arrows in FIG. 1A) that comprises the patent foramen ovale. As stated above, the PFO is a passageway in the septum that has not properly closed. When a PFO is present, the septum primum C typically overlaps the septum secundum D, and the higher pressure in the left atrium B typically closes the flaps of the septum primum C and the septum secundum D so that blood does not leak between the atria A and B. However, when there is a pressure change in the chest, the flaps may separate, thereby permitting blood to leak between the right and left atria. This leakage can result in the migration of unoxygenated blood from the left atrium to mix with the oxygenated blood in the right atrium. Such leakage has been linked to migraine headaches and other maladies described above, such as a stroke or a transient ischemic attack (TIA).

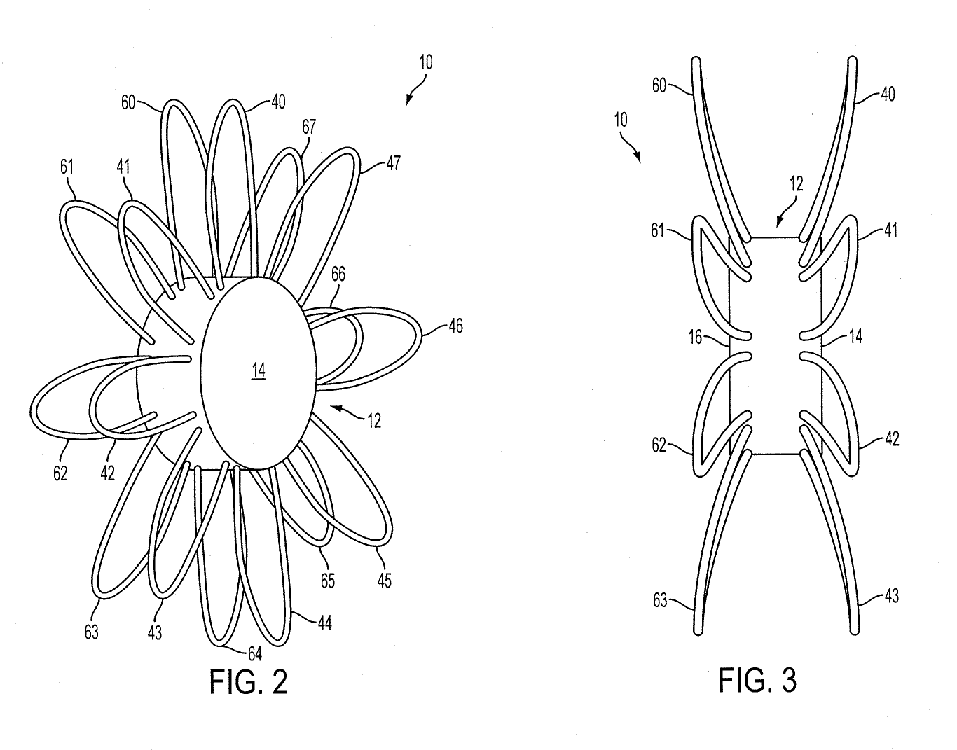

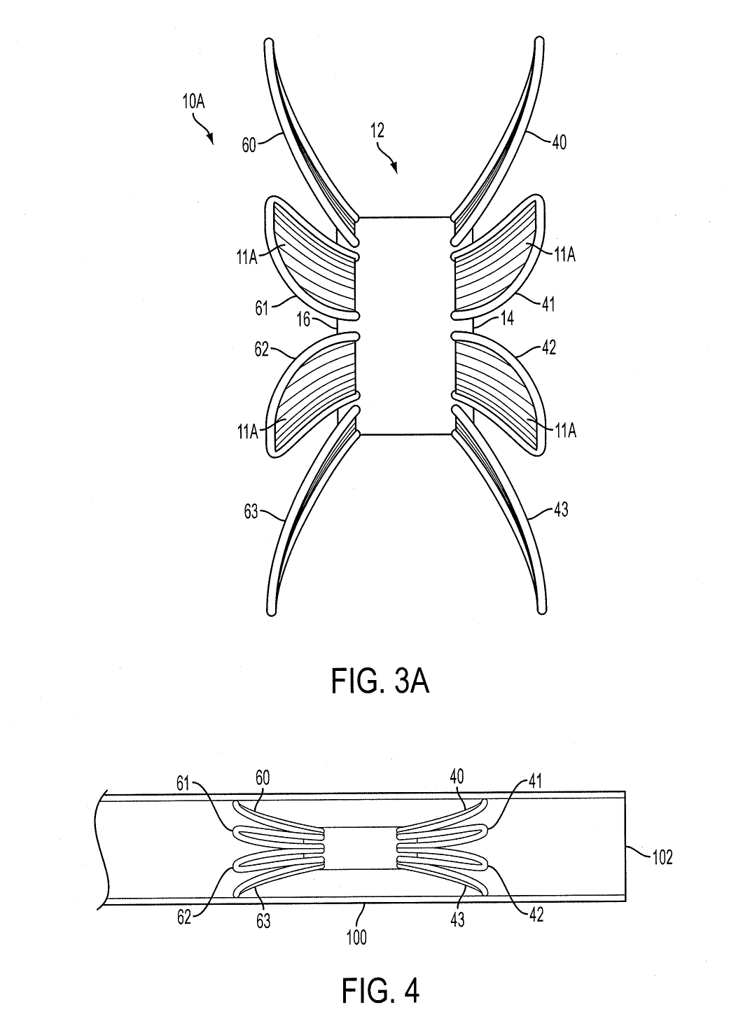

FIG. 2 illustrates a perspective view of a closure device 10 for a body opening, such as a patent foramen ovale, according to one embodiment of the present invention. FIG. 3 illustrates a side view of the closure device of FIG. 2. As illustrated, closure device 10 includes a plug, or core, 12, and a plurality of frame members 40-47 and 60-67 radially extending therefrom. Frame members 40-47 extend circumferentially along the surface adjacent one side, such as the distal side 14 of the plug, and frame members 60-67 extend circumferentially along the surface adjacent an opposite side, such as proximal side 16 of the plug.

In a preferred form, plug 12 comprises a physically modified extracellular matrix (ECM) material. The ECM material may include biotropic or remodelable properties, including in certain forms, angiogenic collagenous extracellular matrix materials. Suitable ECM materials for use in forming plug 12 may include submucosa, renal capsule membrane, abdominal fascia, dermal collagen (including processed dermal collagen from human cadavers, which can be used as allograft in humans), dura mater, pericardium, facia lata, serosa, peritoneum, or basement membrane layers, including liver basement membrane. Suitable submucosa materials may include, e.g., intestinal submucosa, including small intestinal submucosa, stomach submucosa, urinary bladder submucosa, and uterine submucosa. In one example, the plug comprises submucosa derived from a warm-blooded vertebrate. Mammalian submucosa materials are preferred. Porcine submucosa is a particularly preferred material for use herein, particularly porcine small intestine submucosa (SIS), and more particularly small intestine submucosa retaining substantially its native cross-linking. Additional information concerning submucosa and its isolation and treatment, is provided, e.g., in U.S. Pat. Nos. 4,902,508, 5,554,389, 5,993,844, 6,206,931, 6,099,567, and 6,572,650. These patent documents are incorporated by reference herein.

The ECM material will preferably be provided in a compacted, or compressed, condition for delivery to a body passageway. Once positioned within the body passageway, the material is modifiable therefrom to an expanded condition for occluding the passageway. These ECM materials may be prepared and thereby compacted and compressed in a manner suitable for use in the device described herein by methods known in the art. Examples of suitable expandable ECM materials for use herein are described, e.g., in U.S. Pat. No. 8,192,763, U.S. Pat. Publ. No. 2009/0318934, U.S. Pat. Publ. No. 2009/0326577, U.S. Pat. Publ. No. 2009/0317469, and U.S. Pat. Publ. No. 2010/0030246, all incorporated by reference herein. Methods for expanding such materials, and methods for molding or machining such materials into a plug or tube are further described, e.g., in U.S. Pat. Publ. Nos. 2003/0109899, 2005/0085885, 2006/0008419, and 2010/0030246, all incorporated by reference herein. One particularly preferred expandable material comprises expandable small intestine submucosa, or eSIS, as further described in the above-referenced patent documents. In one example, such plug materials may be provided as foam or sponge materials.

As stated, plug 12 preferably comprises a compressed construct, such as the referenced foam or sponge construct, which construct expands after delivery to the target site. Providing a material in an initial compressed condition is desirable when a limited space is available for delivery, such as when the device is positioned in the lumen of a delivery device for transport to the site. Plug 12 may be compressed in a mold or otherwise formed in the desired shape by known means. For example, as described in the incorporated-by-reference patent documents, compact, dense foam or sponge form matrices can be prepared by first hydrating or otherwise wetting a porous sponge matrix, and then compressing and drying the element. Such preparative processes may provide a more dense, rigid and stably compressed sponge matrix than processes such as simple compaction of the dry sponge matrix. Drying can be conducted sufficiently to stabilize the sponge matrix. Compression forces can be applied so as to achieve a final density and/or desirable configuration, and can be applied in one, two or three dimensions, including radially. The drying of the compacted element can involve lyophilization (or freeze drying) or vacuum drying at ambient or elevated temperatures. When processed in this fashion, upon removal of the compaction force, the sponge matrix is stabilized structurally and remains in its highly dense and compacted state until expanded, e.g., by contact with a liquid susceptible to absorption by the matrix.

Upon deployment from the delivery device at the target site, as further described hereinafter, expansion may be carried out, e.g., upon exposure to a liquid medium, such as body fluids. Another suitable fluid comprises a physiologic saline solution (e.g., approximately 0.9% Na). Generally, in intravascular applications, an interventionalist will flush the port with the saline solution prior to, and during, the procedure. Although a saline solution may be preferred for expanding the plug to its fully-expanded configuration, the body fluids (e.g., blood) of the patient would also saturate the plug upon deployment to cause expansion.

Fluid treatment can be used to promote substantial expansion (i.e., greater than about 20% expansion) of the plug. Fluid treatment of the material with, e.g., saline can cause changes in the physical structure of the material which, in turn, causes it to expand. Such changes may include denaturation of the collagen in the material. In certain embodiments, it may be preferred to expand the material to at least about two, three, four, or even more times its original bulk volume. In some examples, the expansion should be at least ten times the crimped (compressed) diameter, with about five to six times the crimped diameter being a favored amount of expansion. In one non-limiting example, the device may be sized such that, upon expansion, it is approximately one to two times larger than the size of a septal opening. As the density of the plug material increases, the material generally cannot expand as much as a less dense plug. Therefore, in such instances, it may not be desirable to crimp the plug material to as low a profile as may theoretically be possible.

It will be apparent to those skilled in the art that the magnitude of the expansion of the plug material may be related to several factors, including, e.g., the concentration or pH of the fluid medium (e.g., an alkaline medium), the exposure time of the fluid medium to the material, and the temperature used in the treatment of the material to be expanded, among others. These factors can be varied through routine experimentation to achieve a material having the desired degree of compression and expansion for a particular application. Examples of such exposure are provided in the incorporated-by-reference patent documents. In addition to the foregoing, one or more known additives may be incorporated into the plug material to promote or accelerate expansion of a compressed plug material. Examples of suitable additives are provided in U.S. Pat. No. 8,329,219, incorporated by reference herein.

The submucosa or other ECM material may also exhibit an angiogenic character, and thus be effective to induce angiogenesis in a host engrafted with the material. Those skilled in the art recognize that the terms “angiogenic” and “angiogenesis” refer to bioactive properties, which may be conferred by a bioremodelable material through the presence of growth factors and the like, which are defined by formation of capillaries or microvessels from existing vasculature in a process necessary for tissue growth, where the microvessels provide transport of oxygen and nutrients to the developing tissues.

The submucosa material or other ECM material may optionally retain and/or otherwise include growth factors or other bioactive components native to the source tissue. For example, the submucosa or other ECM material may retain one or more growth factors such as basic fibroblast growth factor (FGF-2), transforming growth factor beta (TGF-beta), epidermal growth factor (EGF), and/or platelet derived growth factor (PDGF). Submucosa or other ECM material used in certain embodiments of the invention may retain or include other biological materials such as heparin, heparin sulfate, hyaluronic acid, fibronectin and the like. Thus, the submucosa or other ECM material may retain or otherwise include a bioactive component that induces, directly or indirectly, a cellular response such as a change in cell morphology, proliferation, growth, protein or gene expression.

In addition to or as an alternative to the inclusion of native bioactive components, non-native bioactive components such as those synthetically produced by recombinant technology or other methods, may be incorporated into the submucosa or other ECM material, as further set forth in the incorporated-by-reference patent documents cited herein. Submucosa or other ECM material used in certain embodiments of the invention is preferably highly purified as described, for example, in U.S. Pat. No. 6,206,931, incorporated by reference herein.

Frame members 40-47 and 60-67 may be made from a flexible and/or shape memory material. Shape memory materials are well known in the medical arts. Routine practitioners are well aware of the capabilities of such materials, and the manner in which they may be manipulated to recover a desired shape. The nickel-titanium alloy known as nitinol is one preferred material that is commonly utilized in the medical arts for its shape memory and/or superelastic properties. When a frame member is formed from a shape memory material, such as nitinol, the material is typically manipulated into a desired configuration. Once in the desired configuration, the frame member may be annealed, shaped, heated, or otherwise treated in a manner to establish a tendency in the material to return to the desired configuration following manipulation to another configuration.

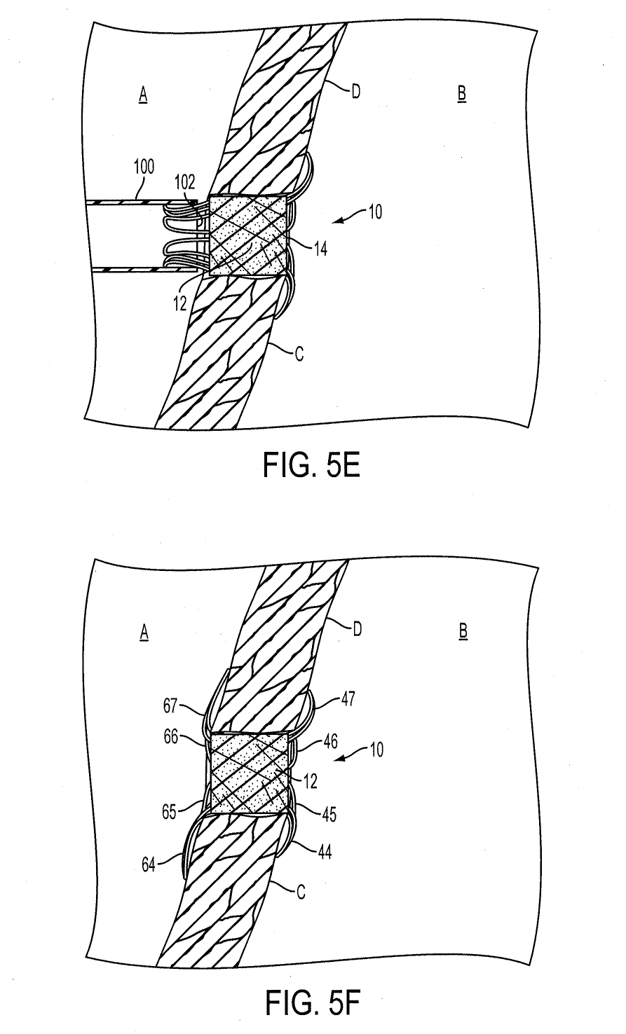

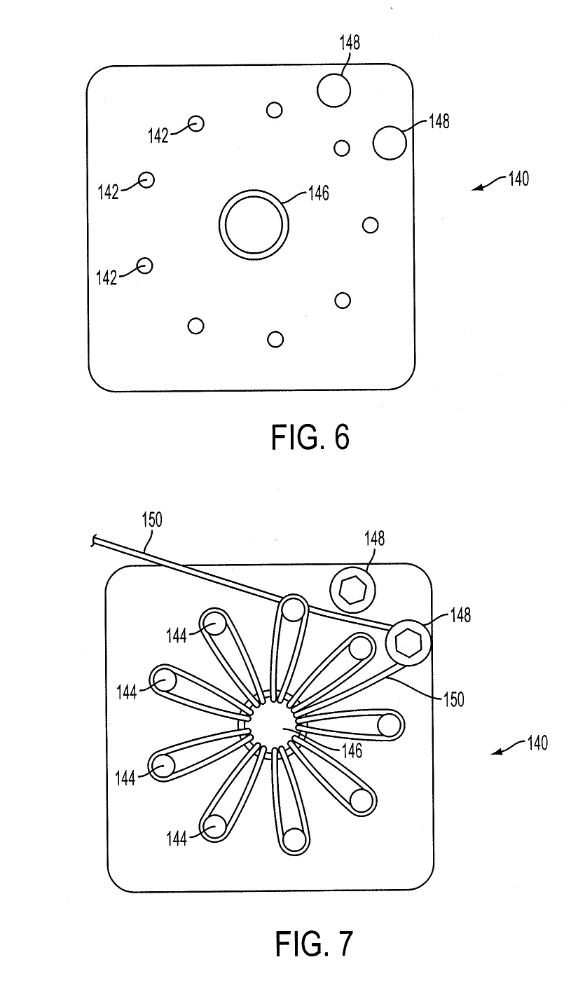

One manner of preparing device 10 of FIG. 3 is described herein. Frame members 40-47 and 60-67 are initially heat set into the desired configuration. Initially, a template 140 formed of a generally rigid material, such as brass or stainless steel, is provided. As shown in the example of FIGS. 6 and 7, template 140 may be generally square-shaped with side dimensions of about 3 cm by 3 cm, and a thickness of about 0.5 mm. The template has a plurality of apertures 142 extending therethrough and arranged in generally circular fashion along the template. The number of apertures corresponds to the number of frame members to be disposed on each side (14, 16) of the plug 12. In the example shown in the figures, eight apertures 142 are provided, to correspond with respective frame members 40-47 and 60-67 on each side of the plug.

A pin 144 extends through each aperture, as shown in FIG. 7. Each pin 144 may be tapped or otherwise securely received in a respective aperture, and arranged therein such that approximately 0.5-1.0 mm of the pin projects outwardly from each side, or face, of template 140. A larger center aperture 146 extends through the center of template 140. Center aperture 146 has a diameter substantially the same as the diameter of the plug 12 in its compressed state. In one example, aperture 146 has a diameter between about 4 and 10 mm, such as between about 6 and 8 mm. A pair of screws 148 extend through template 140. FIG. 7 illustrates the head of screws 148. The threaded body (not shown) of each screw 148 extends to the opposite side of template 140.

As shown in FIG. 7, a continuous wire 150 formed of, e.g., nitinol, having a length of about 150 mm is provided. Generally, wire 150 is wound sequentially around pins 144, e.g., in a manner as described. Wire 150 is initially wound around one of screws 148, and thereafter through aperture 146 and around a pin on the rear face of template 140. The wire is then passed back through central aperture 146 and wound around a pin on the front face of the template (the face shown in FIGS. 6 and 7). The wire is once again passed through central aperture 146 and then wound around another pin on the rear face of the template. This back and forth process is repeated until all of the pins have wire wrapped therearound on each face of the template (FIG. 7). The remaining length of wire 150 may be trimmed away.

The wire is then heat treated while engaged with the template as shown in FIG. 7. Heat treating a shape memory wire in order to enable the treated wire to return to a desired configuration may be carried out by methods well known in the medical arts. For example, the template 140 having the wire 150 wrapped as shown in FIG. 7 may be heated in an oven. The time and temperature to which a wire must be heated to achieve a desired heat setting may vary based upon the size and properties of the particular wire. In one example utilizing nitinol wire, the template and wire are heated to a temperature of about 500 degrees Celsius, e.g., for about ten minutes. Those skilled in the art will appreciate that other well-known heat setting treatments may be substituted. For example, the template and wire may be positioned in a fluidized bed filled with alumina, and maintained in the bed at a predetermined temperature and for a predetermined period of time. Upon removal from the fluidized bed, the template and wire are then immediately quenched, i.e., submerged in a water bath at room temperature in known fashion to assure adequate heat setting. As stated, heat setting a substrate is well known in the medical arts, and still other methods known to those skilled in the art may be substituted for those specifically described herein. Following the heat set, the wire frame may be removed from the template.

A plug 12 of the ECM material, such as the eSIS material described above, of a suitable compressed size may then be inserted into the center aperture portion of the wire frame. Plug 12 will preferably have a dimension sufficient to fill the center aperture (e.g., between about 4 and 10 mm in diameter), and a width sufficient to extend outwardly a short distance (e.g., about 1 mm) from each side thereof. The plug can be attached to the shaped wire frame member (formed from wire 150) by conventional means, e.g., sutures, glue, thermal bonding, etc. As an alternative method of engaging the ECM material to the frame, the ECM (such as eSIS) may be provided in liquid form and injected into the center aperture by known means.

In an alternative embodiment, some or all of respective frame members 40-47 and 60-67 may be provided with a thin membrane or fibrous covering or filler 11A, as shown in FIG. 3A. Covering 11A may be applied to the frame members in the compressed condition by means well known in the medical arts. One manner of applying covering 11A to the frame members is by a process known as electrospinning. In this process, the covering may be formed of any suitable fiber, or material, that is capable of being electrospun onto the frame members, and that is capable of enhancing growth between the covering and/or the frame member, and the adjoining tissue. eSIS, PET (DACRON®), PTFE, and other materials having similar properties are particularly preferred covering materials. Further description of such covering or filler materials, and the electrospinning process, is provided in U.S. Pat. Publ. Nos. 2011/0135806, 2011/0301630, 2012/0022573, and 2012/0259170, all incorporated by reference herein.

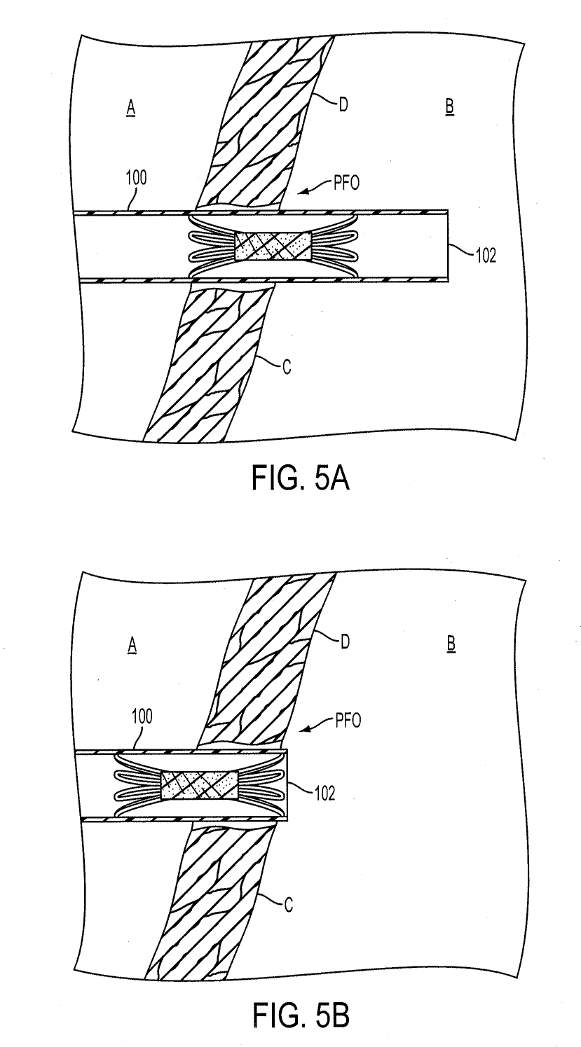

To load device 10 (or 10A) into a delivery device, frame members 40-47 and 60-67 are manually retracted from the position shown in FIG. 3 to a position suitable for loading. FIG. 4 is a side view of device 10 following loading into a delivery catheter 100. Processes for loading a medical device into a delivery catheter, e.g., via pushing or pulling the device into the catheter, and delivery therefrom, are well known in the medical arts. Examples of such loading and delivery are provided, e.g., in incorporated-by-reference U.S. Pat. Publ. Nos. 2009/0062844, 2010/0030246, 2010/0030259, 2010/0234878, and 2010/0312272.

When frame members 40-47 and 60-67 are formed of a shape memory material such as nitinol, the frame members are compressed or otherwise manipulated from a radially expanded position (FIG. 3) into a radially compressed, or restrained, position (FIG. 4) while in the martensitic, or lower temperature, state. When introduced into the body, such materials are warmed to the austenitic state, whereby they revert or otherwise move to the desired, expanded condition. Although nitinol is a particularly preferred material for use in wire 150 herein, those skilled in the art will appreciate that other materials, including other shape memory materials, having similar capabilities are also suitable, and can be substituted for nitinol. Further discussion of shape-memory materials and their use in medical applications are disclosed, e.g., in U.S. Pat. No. 3,012,882, U.S. Pat. No. 3,174,851, U.S. Pat. No. 4,665,906, U.S. Pat. No. 5,108,420, U.S. Pat. No. 5,769,796, U.S. Pat. No. 5,846,247, U.S. Pat. No. 6,451,052, and U.S. Pat. Publ. No. 2009/0062844, all incorporated by reference herein.

As an alternative to the use of shape memory materials, other materials having suitable elasticity properties, such as spring tempered wire, may be substituted. The common property of the compositions useful for forming the frame member is their ability to be manipulated or otherwise arranged into a first shape for loading into a delivery device, and to thereafter move to a desired second shape upon removal of the restraints of the delivery device upon delivery to the target site. Other biocompatible materials capable of such elasticity, manipulation, and/or shape recovery may be substituted for those specifically mentioned hereinabove, all such materials being considered within the scope of the invention.

As shown in FIG. 4, closure device 10 is loaded into delivery catheter 100 for deployment at the target body opening. Delivery catheters for use in deploying a medical device, prosthesis, etc., to a target site in the body of the patient are well known to those of ordinary skill in the medical arts. For example, the delivery catheter can be formed from PTFE (e.g. TEFLON®), a polyamide (e.g. nylon), a polyether block amide (e.g., PEBAX®) or a combination of materials. One example of a suitable delivery catheter includes an inner layer of PTFE, a flat wire stainless steel coil positioned over the PTFE, and a polyamide or polyether block amide outer layer to provide integrity to the overall structure and a smooth outer surface. Such catheters are further described, e.g., in U.S. Pat. No. 5,380,304 and U.S. Pat. Publ. No. 2001/0034514, both incorporated by reference herein. These catheters are commercially available as the FLEXOR® catheter, from Cook Medical Technologies LLC, of Bloomington, Ind. The delivery catheter has an inner diameter sized to allow for the introduction of closure device 10. Illustratively, the inner diameter may range from about 5 to 15 French or more, depending on the size of the closure device and the body passageway intended for closure. Those skilled in the art are believed capable of optimizing such dimensions for closure of a specific passageway.

Radiopaque marker materials and other known visualization enhancement materials may be incorporated into the closure device and/or delivery catheter in known fashion to facilitate radiographic visualization, and/or to render them MRI compatible. One particularly preferred radiopaque material is platinum. Other radiopaque marker materials for use include, but are not limited to, platinum, gold, tungsten, tantalum, tantalum powder, bismuth, bismuth oxychloride, barium, barium sulfate, iodine and the like. Metallic bands of stainless steel, tantalum, platinum, gold, or other suitable materials, can include a dimple pattern, which can further facilitate ultrasound and/or X-ray identification. Radiopaque markers may be incorporated in the closure device and/or the delivery catheter by a variety of common methods, such as adhesive bonding, lamination between two material layers, vapor deposition, and the materials and methods described in U.S. Pat. Publ. No. 2003/0206860, incorporated by reference herein. Alternatively, other visualization or imaging techniques well known in the medical arts, such as ultrasound visualization, may be utilized to visualize the position of closure device 10 and/or delivery catheter 100 in the body of the patient in well-known fashion, such as, by incorporating an echogenic material into the closure device and/or the delivery catheter.

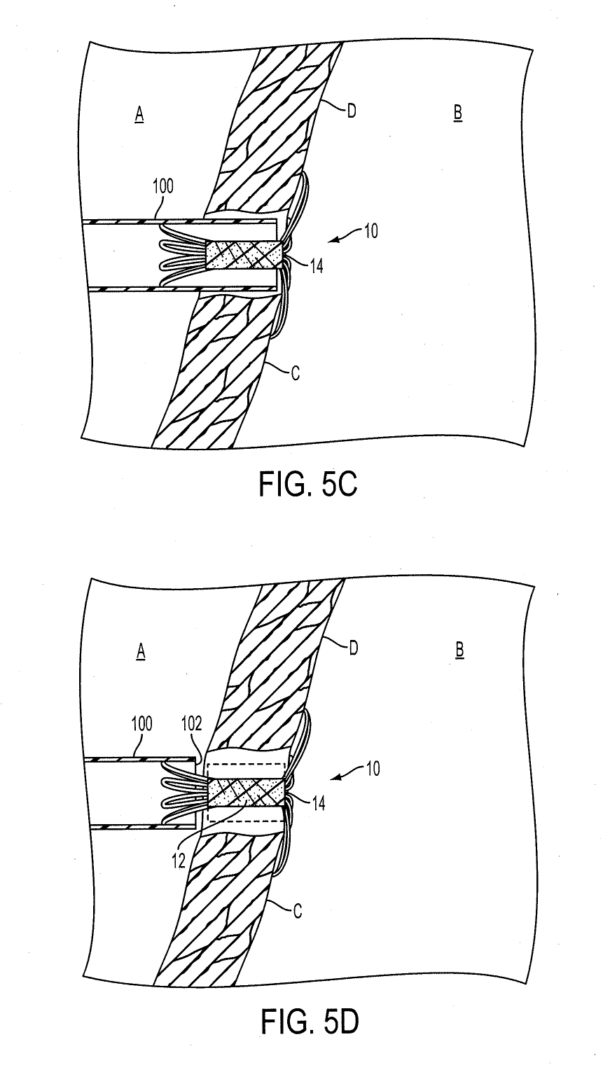

In one aspect, the present invention provides a method for closing or occluding a body opening in a patient. By way of example and not by way of limitation, FIGS. 5A-5F depict a method for closing a patent foramen ovale. In this example, closure device 10 is collapsibly received in delivery catheter 100, as shown in FIG. 4. Closure device 10 may be radially compressed, and thereafter pushed into the delivery catheter in conventional fashion. Alternatively, the closure device can be pulled into the delivery catheter. As stated above, push-pull delivery devices are well-known in the medical arts, and the clinician can readily select an appropriate device for loading and/or delivery. If desired, a delivery system can be provided with a snare/retraction type feature/fixture to enable recapture or reposition the device. One such device that allows retracting/resheathing of the closure device is shown in U.S. Pat. Publ. No. 2010/0168834. Another device that allows delivery, repositioning, and/or removal of the closure device is shown in U.S. Pat. Publ. No. 2013/0090714. Each of the above-referenced patent documents is incorporated by reference herein.

Delivery catheter 100 having closure device 10 collapsibly received therein may be passed into left atrium B across the PFO by known means. For example, delivery catheter 100 may be introduced into the right atrium A by the well-known Seldinger percutaneous entry technique. In the Seldinger technique, a puncture is made by injecting a needle into the entry vessel. A wire guide (not shown) is then inserted through a bore in the needle into the vessel, and the needle is thereafter withdrawn. The wire guide is threaded through the vessel into the right atrium A of the heart, and the delivery catheter is threaded over the wire guide into the right atrium.

The distal end 102 of delivery catheter 100 can be introduced into the left atrium B through the passageway of the PFO that spans septum primum C and septum secundum D, as shown in FIG. 5A. Following introduction of the delivery catheter, the wire guide may be withdrawn from the vasculature in conventional fashion. The delivery catheter is then partially withdrawn, e.g., via one or more well-known medical imaging techniques utilizing marker materials and/or suitable contrast agents injected through the delivery catheter, such that distal end 102 is substantially flush with a distal portion of septum primum C and septum secundum D, as shown in FIG. 5B.

Distal end 14 of closure device 10 may be deployed from the distal end 102 of delivery catheter 100 (FIG. 5C) by well-known means commonly employed in the medical arts for deployment of a device, prosthesis, etc., from a sheath or catheter. Examples of suitable means for the introduction, and deployment, of the closure device are described in the incorporated-by-reference U.S. Pat. Publ. Nos. 2009/0062844, 2010/0030246, 2010/0030259, 2010/0234878, and 2010/0312272. Distal end 14 of closure device 10 can be deployed into the left atrium by withdrawing the delivery catheter 100 from the distal end portion 14 of closure device 10. Alternatively, the distal end of the closure device can be deployed by inserting a conventional pusher device into the proximal end of delivery catheter 100 and pushing the distal end of the closure device from the delivery catheter. As stated, devices for ejecting a medical device from a delivery catheter are well known in the art, and are further described in the patent documents cited above.

Frame members 40-47 of the closure device 10 are self-expanding. Upon the release of frame members 40-47 from the restraint of the delivery catheter in the left atrium, the shape memory or other resilience of the frame members causes frame members 40-47 to revert from the retracted, or compressed, position shown in FIGS. 5A and 5B to the expanded position proximate the PFO opening and the respective septum primum C and septum secundum D, as shown in FIG. 5C.

At this time, the delivery catheter 100 may be further retracted through the opening of the PFO, as shown in FIG. 5D. This movement pulls the expanded frame members 40-47 into juxtaposition with the respective septum primum C and the septum secundum D, and frees plug 12 from the constraints of the delivery catheter. Preferably, frame members 40-47 include curved ends inwardly biased against portions of the septum primum C and the septum secundum D to facilitate more secure anchoring or engagement therewith. By freeing plug 12 from the constraints imposed by the delivery catheter, the plug expands from the diameter as shown in FIG. 5D to occupy the available space in the PFO, as shown in FIG. 5E.

Finally, delivery catheter 100 is fully withdrawn from right atrium A, thereby freeing frame members 60-67. As with frame members 40-47, frame member 60-67 are self-expanding, such that these frame members revert from the retracted, or compressed, position shown in FIGS. 5A to 5D to the expanded position proximate the PFO opening and the respective septum primum C and septum secundum D, as shown in FIG. 5F. Also, as with frame members 40-47, frame members 60-67 preferably include curved ends inwardly biased against portions of the septum primum C and the septum secundum D to facilitate more secure anchoring or engagement therewith.

When fully deployed, a first set of first frame members, including frame members 44 and 45 as shown in FIG. 5F, abuts the septum primum C in the left atrium B, and a second set of first frame members, including frame members 46 and 47 shown in FIG. 5EF abuts the septum secundum D. Similarly, a first set of second frame members, including frame members 64 and 65 abuts the septum primum in the right atrium A, and a second set of second frame members, including frame members 66 and 67 abuts the septum secundum D.

As stated above, plug 12 may be formed in a manner to stimulate angiogenesis with the respective septum primum and septum secundum. In addition, the closure device 10 is preferably sized such that upon implantation and expansion, all or at least a portion of the body passageway (e.g., a PFO) is occluded, whereby the ECM material is stably absorbed and replaced by host tissues.

If desired, a plurality of anchoring members, e.g., barbs, hooks, and like structures (not shown) may be incorporated into the distal ends of the frame members to enhance securement of the structure to the adjacent tissue, and to stimulate tissue response followed by healing. Those skilled in the art can readily select an appropriate type, number and arrangement of anchoring members for use with the inventive closure device. Similarly, those skilled in the art may modify the configuration of the frame members to enhance the suitability of the frame members for engagement with tissue surrounding a particular body passageway.

Preferred embodiments of this invention are described herein. Variations of those preferred embodiments will become apparent to those of ordinary skill in the art upon reading the foregoing description. The inventors expect skilled artisans to employ such variations as appropriate, and the inventors intend for the invention to be practiced otherwise than as specifically described herein. This invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A closure device for a body passageway of a patient, comprising:
a plug member having a first side and a second side, said plug member comprising an extracellular matrix material having a compressed condition, and modifiable therefrom to an expanded condition, the plug member configured to occlude the body passageway in said expanded condition;
a plurality of first frame members disposed generally along said first plug member side, said first frame members aligned in a first position relative to the plug member when said plug member is in said compressed condition, and movable therefrom to a second position; and
a plurality of second frame members disposed generally along said second plug member side, said second frame members aligned in a first position relative to the plug member when said plug member is in said compressed condition, and movable therefrom to a second position.

2. The device of claim 1, wherein said respective frame members are capable of self-movement from said first position to said second position.

3. The device of claim 1, wherein said respective frame members are formed from a shape memory composition capable of self-movement from said first position to said second position.

4. The device of claim 3, wherein said frame members comprise a covering formed thereon, said covering capable of enhancing an engagement of said device with adjacent tissue of said body passageway.

5. The device of claim 1, wherein said plug member comprises an expandable submucosa composition.

6. The device of claim 5, wherein said expandable submucosa composition comprises small intestine submucosa.

7. The device of claim 1, wherein the extracellular matrix material includes at least one growth factor for enhancing tissue growth between said extracellular matrix material and tissue of said body passageway.

8. The device of claim 1, wherein said plug member comprises a cylindrical construct expandable from said compressed condition to said expanded condition upon exposure to a liquid medium.

9. A system for closure of a patent foramen ovale of a patient, the foramen ovale defined by a passageway in the heart of the patient between the septum primum and septum secundum, the system comprising:
a closure device comprising a plug member and a plurality of first and second frame members, said first and second frame members engaged with the plug member and extendable therefrom, the plug member comprising an extracellular matrix material having a compressed condition for deployment along said patent foramen ovale and modifiable therefrom to an expanded condition, the first frame members disposed generally along a first side of the plug member and aligned in a first position relative to the plug member in said compressed condition, said first frame members movable therefrom into a second position relative to the plug member in said expanded condition, the second frame members disposed generally along a second side of said plug member and aligned in a first position relative to the plug member in said compressed condition, said second frame members movable therefrom into a second position relative to the plug member in said expanded condition; and
a delivery device having a lumen for receiving the closure device when the plug member is in the compressed condition and the first and second frame members are in the first position, the closure device deployable from said delivery device for expansion of the plug member to said expanded condition, and for movement of the plurality of first and second frame members to said second position, such that said expanded plug member is positionable along said passageway, said first frame members are positionable in said second position along a first side of said septum primum and said septum secundum, and said second frame members are positionable in said second position along a second side of said septum primum and said septum secundum.

10. The system of claim 9, wherein said frame members are formed from a shape memory composition capable of self-movement from said first position to said second position.

11. The system of claim 10, wherein said frame members comprise segments of a unitary length of said shape memory composition.

12. The system of claim 11, wherein said plug member comprises an expandable submucosa composition.

13. The system of claim 12, wherein said expandable submucosa composition comprises small intestine submucosa.

14. The system of claim 12, wherein said plug member comprises a foam or a sponge construct.

15. The system of claim 9, wherein the extracellular matrix material includes at least one growth factor for enhancing tissue growth between said extracellular matrix material and tissue of said body passageway.

16. A method for closure of a patent foramen ovale of a patient, the foramen ovale defined by a passageway in the heart of the patient between the septum primum and septum secundum, the method comprising:
positioning a closure device in the lumen of a delivery device, the closure device comprising a plug member, and a plurality of first and second frame members engaged with said plug member and extendable therefrom, the plug member comprising an extracellular matrix material having a compressed condition when positioned in the delivery device lumen, said first frame members disposed generally along a first side of said plug member and aligned in a first position relative to the plug member in said delivery device lumen, said second frame members disposed generally along a second side of said plug member and aligned in a first position relative to the plug member in said delivery device lumen;
percutaneously introducing said delivery device having said closure device positioned therein into the right atrium of the heart of a patient;
advancing the delivery device and closure device through the patent foramen ovale such that a distal tip of the delivery device is positioned in the left atrium of the patient;
initiating deployment of said closure device from a distal end of the delivery device such that said first frame members are deployed in the left atrium in the first position and self-movable therefrom to a second position, said first frame members aligned in said second position such that a first set of said first frame members abuts said septum primum and a second set of said first frame members abuts said septum secundum in said left atrium;
withdrawing the delivery device relative to the closure device such that the plug member is deployed intermediate the septum primum and the septum secundum, the plug member movable from said compressed condition to an expanded condition in said passageway; and
further withdrawing the delivery device relative to the closure device such that the second fame members are deployed from the delivery sheath in the right atrium in the first position and self-movable therefrom to a second position, said second frame members aligned in said second position such that a first set of said second frame members abuts said septum primum and a second set of said first frame members abuts said septum secundum in said right atrium.

17. The method of claim 16, wherein said extracellular matrix material is movable into the expanded condition upon exposure to a liquid medium.

18. The method of claim 16, wherein said extracellular matrix material includes one or more growth factors for enhancing tissue growth between said expanded extracellular matrix material and adjoining tissue of said septum primum and said septum secundum in said passageway.

19. The method of claim 16, wherein said extracellular matrix material of said plug member comprises a submucosa composition comprising one or more components capable of enhancing growth between said plug member in the expanded condition and adjacent tissue of said passageway.

20. The method of claim 17, wherein said respective frame members are formed from a unitary length of a shape memory composition.
